(19) United States
(12) Patent Application Publication
(10) Pub. No.: US 2023/0105767 A1
(43) Pub. Date: Apr. 6, 2023

(54) ANNOTATING AND MANAGING OF THERAPEUTIC OR BIOLOGICAL DIGITAL DATA

(71) Applicant: Janssen Biotech, Inc., Horsham, PA (US)

(72) Inventors: Weiwei Schultz, Furlong, PA (US); Aleksandar Stojmirovic, North Wales, PA (US)

(21) Appl. No.: 17/907,235

(22) PCT Filed: Mar. 25, 2021
(86) PCT No.: PCT/US2021/024102

§ 371 Date: Sep. 23, 2022

Related U.S. Application Data
(60) Provisional application No. 63/000,360, filed on Mar. 26, 2020.
(60) Provisional application No. 63/000,350, filed on Mar. 26, 2020.
(60) Provisional application No. 63/000,330, filed on Mar. 26, 2020.
(60) Provisional application No. 63/000,367, filed on Mar. 26, 2020.

(51) Int. Cl. G16B 50/10; G16B 50/30; G16H 10/20

(52) U.S. Cl. CPC G16B 50/10; G16B 50/30; G16H 10/20

(57) ABSTRACT

Systems, system integrations, non-transitory computer program products, and methods are described for managing digital data including therapeutic digital data or biological digital data. Such systems include at least one data processor and memory storing instructions, which when executed by at least one computing device result various operations. The digital data uploaded via a pre-defined pathway is received. The digital data is annotated with metadata based on a pre-defined annotation schema associated with the pre-defined pathway. The metadata facilitates storage and identification of the annotated digital data in a permanent data repository. Data encapsulating a notification of completion of the annotating is provided for further storage and analysis.

### PRIORITY CLAIMS

This application claims priority to (i) U.S. Application No. 63/000,367, filed Mar. 26, 2020, entitled “Processes for Enhanced Biological Digital Data Utilization,” (ii) U.S. Application No. 63/000,360, filed Mar. 26, 2020, entitled “Materials and Methods for Improved Management of Therapeutic Digital Data,” (iii) U.S. Application No. 63/000,330, filed Mar. 26, 2020, entitled “Systems and System Integrations for Biological Digital Data Utilization,” and (iv) U.S. Application No. 63/000,350, filed Mar. 26, 2020, entitled “Non-transitory Machine Program Product Storing Instructions for Biological Digital Data,” the contents of each of which are incorporated herein in their entirety.

### FIELD

The subject matter described herein relates to enhanced techniques for annotating and managing therapeutic and/or biological digital data.

### BACKGROUND

Data is a vital organizational asset. However, when not managed properly, it can accumulate as unutilized digital storage, without being utilized to its full potential of being re-used in future research contexts. Complex daily workflows using this data frequently rely on multiple disparate systems, piece-mealed together by specialized ad-hoc toolsets. Such architecture can create disjointed user experiences.

### SUMMARY

In one aspect, a method for managing therapeutic and/or biological digital data includes receiving therapeutic and/or biological digital data uploaded via a pre-defined pathway. The therapeutic and/or biological digital data is annotated with metadata based on a pre-defined annotation schema associated with the pre-defined pathway. The metadata facilitates storage and identification of the annotated therapeutic and/or biological digital data in a permanent data repository. Data encapsulating a notification of completion of the annotating for further storage and analysis is provided.

In some variations, the metadata can include at least one mandatory study field that describes at least one of (i) a therapeutic study identification (ii) a therapeutic study type defining a type of study in drug development, preclinical research, or a clinical trial, (ii) a therapeutic study name, (iii) a therapeutic study description defining the study objectives, protocol, or design, (iv) an organism under study, or (v) a submitter.

In other variations, the metadata can include at least one mandatory experiment field that describes at least one of (i) a therapeutic study identification, (ii) an experiment tag, (iii) an experiment description, (iv) a measurement type, (v) a technology type defining a detection method or technology used to conduct an experiment, (vi) a platform defining a version of the technology type used to conduct the experiment, (vii) a contributor, (viii) a contact defining a primary point of contact for the therapeutic and/or biological digital data, or (ix) a submitter of the therapeutic and/or biological digital data.

In some variations, the metadata can include at least one optional study field that describes at least one of (i) a study intervention defining a compound or a molecule under study, (ii) a disease under study, (iii) a therapeutic area, (iv) a functional area, (v) a disease area stronghold, (vi) a pathway area stronghold, (vii) a keyword, or (viii) an electronic lab notebook number.

In other variations, the metadata can include at least one optional experiment field that describes at least one of (i) a related study identifier, (ii) an atomical entity defining where samples for an experiment originated, (iii) a cell type classification, (iv) cell line information, (v) a sample acquisition method defining a method or a procedure used to acquire a sample, (vi) a disease under study, (vii) sample disease activity defining status of a disease of the sample, (viii) sample treatment defining an agent used to treat the sample, (ix) a time point defining a sample collection time point, (x) a species under study, (xi) a host species defining a host organism for the study, (xii) a number of sample taken for the experiment, (xiii) a method used to generate the therapeutic and/or biological digital data, (xiv) a keyword associated with the therapeutic and/or biological digital data, (xv) a rights statement, (xvi) a rights holder, (xvii) a creation location defining a location where the therapeutic and/or biological digital data was generated, or (xviii) a contributor to the therapeutic and/or biological digital data.

In some variations, the annotating can include determining a data format of the therapeutic and/or biological digital data. Based on the data format, the therapeutic and/or biological digital data can be consolidated and converted to a parsable, human readable text file format. The metadata can be assigned to the parsable, human readable text file format.

In other variations, the therapeutic and/or biological digital data can be transferred and stored in a read-only format to the permanent data repository.

In some variations, the pre-defined pathway can point to a hierarchical data folder in an intermediary data repository. The metadata can be associated with the hierarchical folder.

In other variations, the notification can inform an administrator to transfer the therapeutic and/or biological digital data to the permanent data repository.

In some variations, data stored in the permanent data repository cannot be modified, deleted, or overwritten.

In other variations, the therapeutic and/or biological digital data can be provided in a read-only format to a graphical user interface for inspection.

In some variations, the metadata can be defined by a user when uploading the therapeutic and/or biological digital data using the pre-defined pathway.

In other variations, the therapeutic and/or biological digital data can include biological digital data having at least one of bio therapeutic data, biotechnological data, molecular data, biomarker data, transcriptional data, phenome data, or image data.

In another aspect, a system for managing therapeutic and/or biological digital data includes means for receiving uploaded therapeutic and/or biological digital data, means for annotating the therapeutic and/or biological digital data with metadata, and means for providing a notification of completion of the annotating for further storage and analysis. The metadata facilitates storage and identification of the annotated therapeutic and/or biological digital data.

In yet another aspect, a system for managing therapeutic and/or biological digital data includes at least one data processor and memory storing instructions, which when executed by at least one computing device result in operations such as receiving therapeutic and/or biological digital data uploaded via a pre-defined pathway. The therapeutic and/or biological digital data is annotated with metadata based on a pre-defined annotation schema associated with the pre-defined pathway. The metadata facilitates storage and identification of the annotated therapeutic and/or biological digital data in a permanent data repository. Data encapsulating a notification of completion of the annotating for further storage and analysis is provided.

The systems can be computer systems that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The subject matter described herein provides many technical advantages. For example, the current subject matter enables efficient storage and consistent retrieval of data in a FAIR (Findable, Accessible, Integrated, Reproducible) manner by enforcing controlled data movement and annotation workflows. Use of the current subject matter provides a scalable enterprise-grade solution to managing therapeutic and/or biological digital data. Additionally, using the subject matter described herein, data can be more easily found, integrated, and/or shared within the biological field so as to rapidly deliver new insights.

### BRIEF DESCRIPTION OF THE DRAWINGS

FIG. 1 illustrates an example system that includes a client-server architecture.

FIG. 2 illustrates an example folder hierarchical structure in which the therapeutic and/or biological digital data can be organized within.

FIG. 3 illustrates an example table of study fields can be provided as input to the BDM/TDM module.

FIG. 4 illustrates an example table of experiment fields can be provided as input to the BDM/TDM module.

FIG. 5 is a flow chart illustrating a method for managing therapeutic and/or biological digital data.

FIG. 6 is a diagram illustrating a sample computing device architecture for implementing various aspects described herein.

Like reference symbols in the various drawings indicate like elements.

### DETAILED DESCRIPTION

The subject matter described herein relates to computer-based therapeutic and/or biological digital data management that provides for enhanced dataflow of raw research data from initial ingestion to the storage of analysis-ready datasets. More specifically, a computer-based workflow-specific platform as described can offer end-to-end data ingestion, storage, and/or data retrieval capabilities from internal scientific instruments and/or external vendors. Intuitive dashboards (i.e., graphical user interfaces) can alert users to the status of incoming data and can guide them on further actions to take, such as data annotation. Templates can be provided for annotating data with study-level metadata. Design tables detailing study cohorts and analysis parameters can also be coupled with the raw data as to provide contextual information about the experiment when accessed in the future.

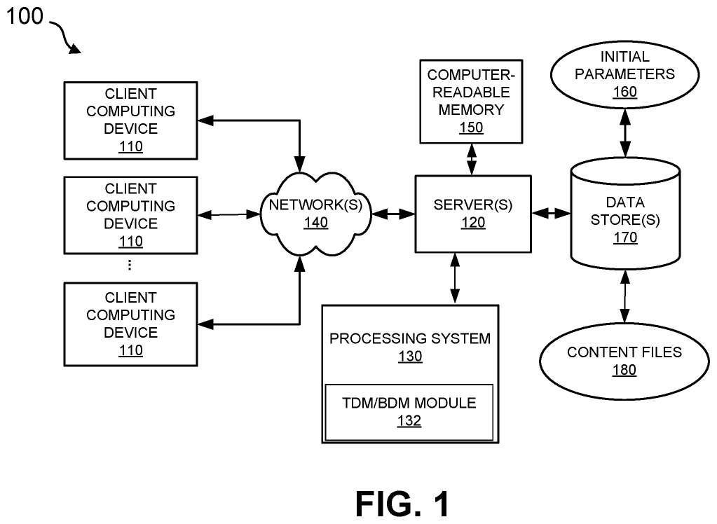

FIG. 1 illustrates an example system 100 that includes a client-server architecture. One or more client computing devices 110 access one or more servers 120 running a biological data management (BDM) or therapeutic data management (TDM) (BDM/TDM) module 132 on a processing system 130 via one or more networks 140. The one or more servers 120 may access a computer-readable memory 150 as well as one or more data stores 170. The one or more data stores 170 may include initial parameters 160 as well as content files 180. Computer-readable memories 150 or data store(s) 170 may include one or more data structures for storing and associating various data used in the example systems for managing therapeutic and/or biological digital data. For example, a data structure stored in any of the aforementioned locations may be used to store data from XML files, initial item parameters, and/or data for other variables described herein.

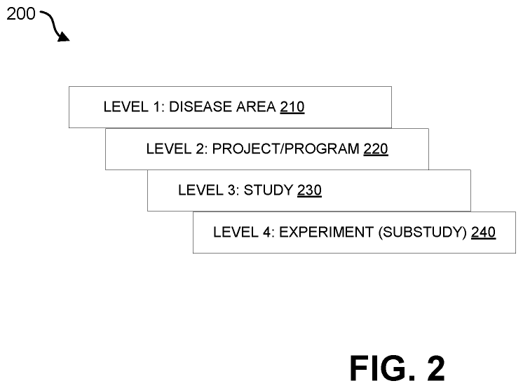

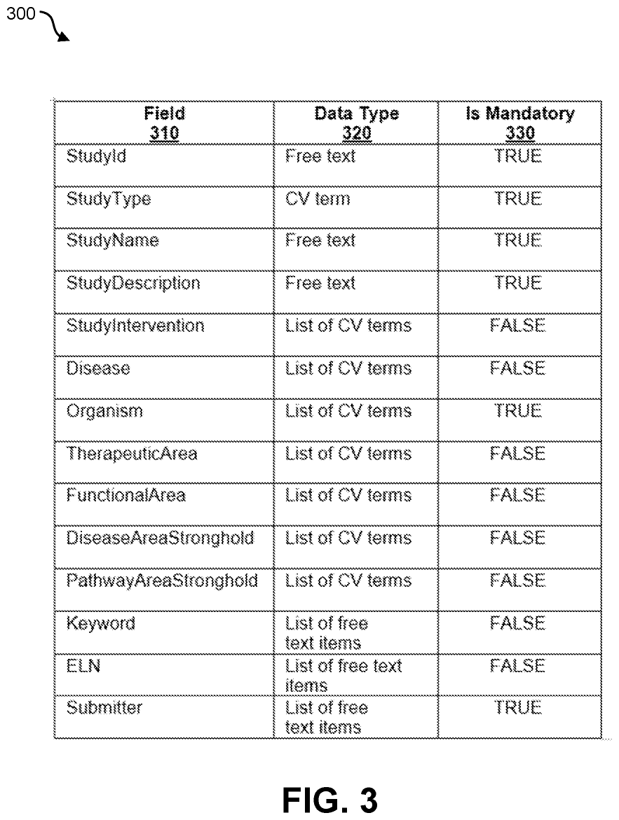

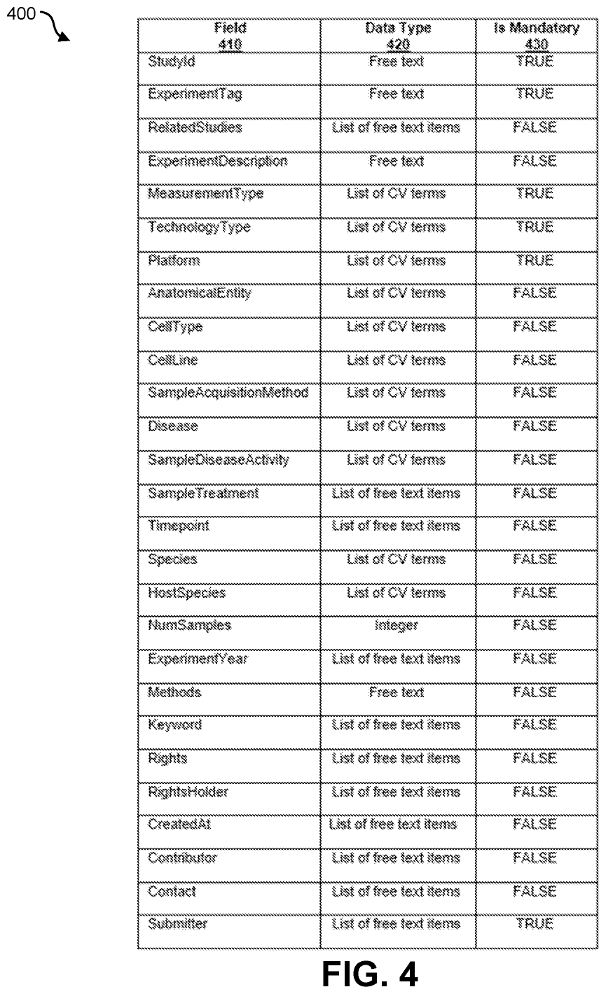

Therapeutic and/or biological digital data can be transferred from one or more client computing devices 110 via network(s) 140 to BDM/TDM module 132 via server(s) 120. Therapeutic and/or biological digital data includes biological digital data. This biological digital data can include, but is not limited to, bio therapeutic data, biotechnological data, molecular data, biomarker data, transcriptional data, phenome data, image data, and the like. The client computing devices 110 can be any type of computing device that can capture, collect, and/or transmit the therapeutic and/or biological digital data. For example, client computing devices 110 can be a data collection instruments, mobile devices, personal computers, and the like. The therapeutic and/or biological digital data can be uploaded to remote devices (e.g., server(s) 120, processing system 130, computer-readable memory 150, data store(s) 170), for example, using the pre-defined pathway. The pre-defined pathway can be generated by the BDM/TDM module 132. The pre-defined pathway (e.g., unique URL) can be digital pointer that specifies a data hierarchy within a permanent repository (e.g., data store(s) 170). A notification can be transmitted by client computing device 110 to the BDM/TDM module 132 to notify the BDM/TDM module 132 that the therapeutic and/or biological digital data is uploaded and ready for transferring to a permanent repository. The therapeutic and/or biological digital data can be stored within a permanent repository (e.g., data store(s) 170) utilizing a digital data hierarchy such as the one described in more detail in FIG. 2. Prior to storage, the uploaded therapeutic and/or biological digital data can be annotated (e.g., semi-automatically or automatically with no human intervention based on the pre-defined pathway) with metadata. That metadata can be defined by a series of mandatory and/or optional fields defined by the client computing device 110 on data upload. These fields establish a pre-defined schema that can be utilized to define annotations for the uploaded data. Such fields are described in more detail below in the descriptions of FIGS. 3-4. It is the metadata that can facilitate storage and identification of the annotated therapeutic and/or biological digital data in a permanent data repository (e.g., data store(s) 170). Once the data is uploaded and annotated, a notification can be generated by the client computing device 110 and provided or transmitted to the BDM/TDM module 132. The notification can be displayed on a graphical user interface of processing system 130. The notification can be loaded into memory such as computer-readable memory 150. The notification can be stored into data storage such as data store(s) 170. The notification can be transmitted to a remote computing system such as processing system 130 and/or server(s) 120 via network(s) 140.

Upon receiving the notification, the annotated therapeutic and/or biological digital data can be transferred from the BDM/TDM module 132 to the data store(s) 170. The data can be stored in the standard hierarchy described in more detail in FIG. 2, to facilitate publishing. The data within data store(s) 170 can be accessed for further analysis, but cannot be modified, deleted, or overwritten (e.g., stored it read-only format). The data within data store(s) 170 can be published such that it is in a readable format.

FIG. 2 illustrates an example folder hierarchical structure 200 in which the therapeutic and/or biological digital data can be organized within. The therapeutic and/or biological digital data can be organized within a number of different levels of the folder hierarchical structure 200. For example, the folder hierarchical structure 200 can include level 1 relating to a disease or disease area 210 (e.g., inflammatory bowel disease (IBD)), level 2 relating to a project or program 220 (e.g., Mount Sinai School of Medicine (MSSM)—collaboration), level 3 relating to a study 230 (e.g., Mount Sinai Crohn's and Colitis Registry (MSCCR-CrossSectional)), and an experiment or sub study 240 (e.g., tissue/measurement type, computational, clinical, biopsy-ribonucleic acid (RNA), whole blood m-RNA (WB-mRNA)).

FIG. 3 illustrates an example table of study fields 310 can be provided as input to the BDM/TDM module 132. Each of the study field 310 has a corresponding data type 320 that may be accepted as input by the user. Additionally, each study field 310 can be denoted as either mandatory or optional 330. A biological study identification is an example study field 310 which can be a unique identifier of a study being performed. The biological study identification can correspond with Level 3: Study 230 of a hierarchical structure 200. A study type is another example study field 310 which defines a type of study such as drug development, preclinical research, and/or clinical trials. There are a number of different types of studies that may be provided for this study field 310 which can include, but are not limited to, an in silico study, an in vitro study, an in vivo study, a Phase 0 trial, a Phase I trial, a Phase I/II trial, a Phase II trial, a Phase II/III trial, a Phase III trial, a Phase Ma trial, a Phase IIIb trial, a Phase IIa trial, a Phase IIb trial, a Phase IV trial, a Preclinical study, an observational study, a Phase Ia trial, a Phase Ib trial, an Ex vivo study, a Comparative study, or a Meta-analysis.

A study name and study description are example study fields 310. A study name can be the official title of a trial or name commonly used to refer to the study. A study description can be a description of the study's objectives, protocols, and/or design.

A study intervention is another example study field 310 which defines the compound or molecule under study, including placebo or alternative treatment. The study intervention field can be used solely for clinical trial or a study that contains samples from human patients. In some cases, this field may be left blank such as for Preclinical, in-vitro, and/or other studies. Example study intervention fields can include VE303, Vedolizumab, Placebo, Ustekinumab, Guselkumab, Adalimumab, Golimumab, Etanercept, Peficitinib, Infliximab, TD-1473, ASO, Methotrexate, Sirukumab, Daratumumab, and/or Secukinumab.

Another example study field 310 is a disease under study as defined by the Human Disease Ontology (DOID). Such diseases can include, but are not limited to, an acquired metabolic disease, Alzheimer's disease, Ankylosing spondylitis, arthritis, asthma, an autoimmune disease of central nervous system, an autoimmune disease of the nervous system, an autoimmune hypersensitivity disease, a bone disease, a bone inflammation disease, a bronchial disease, a central nervous system disease, a chronic obstructive pulmonary disease, Clostridium difficile colitis, colitis, connective tissue disease, Crohn's disease, Demyelinating disease, a disease of anatomical entity, a disease of metabolism, fatty liver disease, gastrointestinal system disease, healthy (e.g., no disease), hypersensitivity reaction disease, hypersensitivity reaction type IV disease, inflammatory bowel disease, inherited metabolic disorder, integumentary system disease, intestinal disease, kidney disease, lipid storage disease, lower respiratory tract disease, lung disease, lupus erythematosus, lysosomal storage disease, morbid obesity, multiple sclerosis, musculoskeletal system disease, nervous system disease, neurodegenerative disease, nonalcoholic fatty liver disease, nutrition disease, obesity, obstructive lung disease, over nutrition, psoriasis, psoriatic arthritis, respiratory system disease, rheumatoid arthritis, sarcoidosis, skin disease, skin sarcoidosis, syndrome, tauopathy, ulcerative colitis, urinary system disease, Celiac disease, Systemic Lupus Erythematosus, Lupus Nephritis, primary biliary cirrhosis, juvenile idiopathic arthritis, Cutaneous lupus erythematosus, Chronic obstructive pulmonary disease, Scleroderma, Atopic dermatitis, Ichthyosis vulgaris, Non-IBD controls, Osteoarthritis, alopecia, Arthralgia, Hidradenitis, Type 1 Diabetes Mellitus, and/or Sjogren's syndrome. The disease field may be left blank for any study that does not have samples from human patients. For clinical studies, the disease field can be annotated only with the disease under study and an individual experiment annotation can indicate that control samples are added.

Another example study field 310 include an organism under study. Such organisms can include, but are not limited to, Homo sapiens, Human gut metagenome, unclassified sequences, Canis lupus familiaris, Mus musculus, mouse gut metagenome, Clostridiales bacterium VE202-01, Clostridiales bacterium VE202-03, Hungatella hathewayi VE202-04, Clostridiales bacterium VE202-06, Clostridiales bacterium VE202-07, Clostridiales bacterium VE202-08, Clostridiales bacterium VE202-09, Clostridiales bacterium VE202-13, Clostridiales bacterium VE202-14, Clostridiales bacterium VE202-15, Clostridiales bacterium VE202-16, Clostridiales bacterium VE202-18, Clostridiales bacterium VE202-21, Clostridiales bacterium VE202-26, Clostridiales bacterium VE202-27, Clostridiales bacterium VE202-28, Clostridiales bacterium VE202-29, and/or Rattus norvegicus.

Another example study field 310 include a therapeutic area. Therapeutic areas can include, but are not limited to, cardiovascular and metabolism, immunology, clinical immunology, infectious diseases and vaccines, neuroscience and pain, oncology, pulmonary hypertension, and/or pulmonary arterial hypertension.

A functional area is another example study field 310. Example functional areas include, but are not limited to, bio therapeutics, bio therapeutics development, clinical supply chain, computational sciences, discovery and manufacturing sciences, discovery sciences, disease interception accelerator, external innovation, global clinical development, global public health, global regulatory affairs, Janssen human microbiome institute, Janssen prevention center, Janssen research and development, quantitative sciences, small molecule development, and/or statistics and decision sciences.

A disease area stronghold (DAS) is another example study field 310. Example disease area stronghold CV terms can include, but are not limited to, bacterial vaccines DAS, Hepatitis DAS, IBD DAS, metabolism DAS, mood DAS, neurodegeneration DAS, oncology driver mutation DAS, pulmonary arterial hypertension (PAH) DAS, prostate cancer DAS, respiratory infection DAS, retinal disease DAS, rheumatology DAS, thrombosis DAS, viral vaccines DAS, and/or immuno-dermatology DAS.

Another example study field 310 can include a pathway area stronghold (PAS). Such a field can include glutamate PAS, interleukin (IL)-23 PAS, and/or Immuno-oncology PAS. Other example study fields 310 include a keyword, an ELN, and/or a submitter. A keyword can define a keyword or phrase used for text-searching. The submitter can be an individual who was responsible for uploading the study data as described in detail in FIGS. 1-2.

Each study field 310 can be associated with a various data type 320. For example, some study fields 310 can be free text (e.g., biological study identification, a study name, and/or a study description) or a list of free text items (e.g., keyword, electronic lab notebook (ELN) number, and/or submitter). Some study fields can be control vocabulary (CV) terms (e.g., study type) or a listing of CV terms (e.g., study intervention, disease, organism, a therapeutic area, functional area, disease area stronghold, pathway area stronghold).

Some study fields 310 can be mandatory such that a user must enter data associated with these fields when uploading digital data. Other study fields 310 can be optional fields of data that may be entered at the discretion of the user uploading the therapeutic and/or biological digital data. Example study fields 310 that are mandatory can include a study identification, a study type, a study name, a study description, an organism, and/or a submitter. Example optional fields can include a study intervention, a disease, a therapeutic area, a functional area, a disease area stronghold, a pathway area stronghold, a keyword, and/or an ELN number.

FIG. 4 illustrates an example table of experiment fields 410 can be provided as input to the BDM/TDM module 132. Each of the experiment field 410 has a corresponding data type 420 that may be accepted as input by the user. Additionally, each experiment field 410 can be denoted as either mandatory or optional 430. A biological study identification is an example experiment field 410 which can be a unique identifier of a study being performed. The biological study identification can correspond with Level 3: Study 230 of a hierarchical structure 200. An experiment tag is another example experiment field 410 that can define a name and/or type of experiment. The experiment tag can correspond with Level 4: Experiment (Sub study) 240 of a hierarchical structure 200.

Related studies is another example experiment field 410 that defines a list of secondary study identifiers. Each study identifier can correspond to the Level 3: Study 230 of a hierarchical structure 200. The related study field can be used when an experiment contains or refers to data or samples from multiple studies. For example, the tissue samples from several distinct clinical trials may be run and analyzed together. In this case, one study should be chosen as the main parent study in the hierarchy (e.g., indicated through the study identification), while the remaining studies can be indicated through the related study field.

An experiment description is another example experiment field 410 that is a textual description of relevant information about the experiment. Another example experiment field 410 is a measurement type which defines what is being measured or analyzed. Example measurement type CV terms can include transcriptional profile, metagenomics, clinical observations, genotyping, metatranscriptomics, computational processing, protein expression profiling, histology, immunophenotyping, cell counting, epigenetics, diagnostic procedure, immunostaining, deoxyribonucleic acid (DNA) sequencing, colonoscopy, imaging, and/or B-cell receptor (BCR) sequencing.

A technology type is another example experiment field 410 that defines a detection method, technology, and/or assay used during the experiment. Example CV terms for the technology type include 454 sequencing, Applied Biosystems (ABI) Sequencing by Oligonucleotide Ligation and Detection (SOLiD) sequencing, assay by high throughput sequencer, assay by mass spectrometry, assay by sequencer, chromatin immunoprecipitation (ChIP)-seq, DNA analysis, DNA microarray, DNA methylation analysis, DNA microarray analysis, DNA-seq, Exome sequencing, Gene expression analysis, IP-seq, Illumina sequencing, large-scale sequencing, MicroRNA expression profile, microarray analysis, molecular profiling, mutation detection, nucleic acid sequencing, polymorphism analysis, protein analysis, protein expression analysis, protein sequencing, proteomic profiling by mass spectrometer, RNA-seq of non-coding RNA, transcription profiling by high throughput sequencing, whole genome association study, whole genome sequencing, whole genome shotgun sequencing, complementary DNA (cDNA) expression, cDNA microarray analysis, mRNA sequencing, microRNA profiling by high throughput sequencing, RNA-seq, clinical observations, computational analysis, single nucleotide polymorphisms (SNP) array, 16S rRNA amplification and sequencing, sequence alignment, lab tests, flow cytometry, immunoassay, immunohistochemistry, imaging, Illumina Global Screening Array, whole exome sequencing, expression quantitative trait locus (eQTL) analysis, quantitative polymerase chain reaction (qPCR), allele-specific PCR, cell counting, diagnostic procedure, mass cytometry, and/or whole slide imaging.

A platform is another example experiment field 410 that defines a specific version (e.g., manufacturer, model, etc.) of a technology that is used to carry out the experiment. Example CV terms for the platform include, but are not limited to, a 454 Genome sequencer FLX, an AB SOLiD system, a cytometer, a DNA Sequencer, a FACSAria, a flow cytometer, a flow cytometer sorter, Illumina HiSeq 4000, SOLiD 4 system, Illumina HiSeq, Illumina NextSeq, Illumina NextSeq 500, HGU133Plus2, hugene10st, Illumina HiSeq 2000, Illumina HiSeq 2500, clinical observations, computational analysis, Illumina Infinium, Immunochip, Illumina MiSeq, high performance computing (HPC) cluster, HTMG430PM, Q-Exactive (Thermo), Orbitrap XL, hugene21st, Singulex, Luminex, MSD, Millipore, SomaLogic, Immunoassay, Immunohistochemistry, enzyme-linked immunosorbent assay (ELISA), blood test, lipid panel, endoscopy, TaqMan, real-time PCR, allele-specific PCR, Affymetrix miRNA1.0, Affymetrix miRNA2.0, eQTL analysis, Epiontis, Illumina Global Screening Array, whole exome sequencing, Illumina Novaseq, Hemocytometer, Niox Mino, PBL, MRC-5 cell, Pantomics, OpenArray, Theranos, high-performance liquid chromatography (HPLC), Mass spectrometer, Illumina Genome Analyzer IIx, mogene21st, mass cytometry, Illumina Infinium ImmunoArray-24 v2 BeadChip, Illumina Infinium Multi-Ethnic Genotyping Array (MEGAEX), AB 5500xl-W Genetic Analysis System, Ion Torrent S5, Infinium MethylationEPIC, Fluidigm Biomark HD, SMART-Seq, Illumina SBS Kit v3 (200 Cycles), Bisulfite Sequencing, and/or Aperio.

Another example experiment field 410 is an anatomical entity that defines where samples for the experiment originated. Example anatomical entities include, but are not limited to, ileum, colon, stool, duodenum, spleen, synovium, whole blood, kidney, serum, skin, rectum, mucosa, urine, sputum, lung, nasal lavage, bronchus, plasma, buccal surface, hair follicle, bronchoalveolar lavage, spflex, cecum, ileocecal valve, paw, peripheral blood mononuclear cell (PBMC), small intestine, synovial fluid, liver, and/or salivary gland.

A cell type is another example experiment field 410 that defines a cell type classification. Example CV terms for cell types can include, but are not limited to, an animal cell, a cell in vitro, a cell line cell, a circulating cell, a cultured cell, an Epithelial cell, an Eukaryotic cell, an experimentally modified cell in vitro, a hematopoietic cell, an immortal cell line cell, a Leukocyte, a mononuclear cell, a mortal cell line cell, a native cell, a nongranular leukocyte, a peripheral blood mononuclear cell, a primary cultured cell, an immature dendritic cell (iDC), a keratinocyte, a bronchial epithelial cell, a Jurkat cell, a T cell, a B cell, a regulatory T cell, a Th17 cell, a T/B/NK cell, a monocyte, a squamous epithelial cell, a macrophage, a polymorphonuclear leukocyte, an eosinophil, a lymphocyte, and NK cell, a dendritic cell, a Basophil, a Granulocyte, a CD14+ monocyte, a NKT cell, a Plasmacytoid dendritic cell, a Fibroblast-like synoviocyte, a CD38+ cell, a Synovial fluid mononuclear cell, an Endothelial cell, a smooth muscle cell, an innate lymphoid cell, a Th2 cell, a Th1 cell, a Treg cell, a CD64 dendritic cell, and/or a skin fibroblast.

Another example experiment field 410 includes a sample acquisition method which defines a method or procedure used to acquire a sample. Example CV terms for the sample acquisition method can include, but are not limited to, a biopsy, a surgical resection, a surface swab, and/or brushing.

A disease is another example experiment field 410 that defines a disease under study. This field was previously explained in relation to the example study field 310. This disease information can be inherited from the information provide within the biological study information, which was previously described in detail.

Another example experiment field 410 includes a sample disease activity which defines any inflammation or disease status of a sample. Example CV terms associated with the sample disease activity can include, but are not limited to, healthy, inflamed, lesion, non-lesion, non-inflamed, normal, involved, or uninvolved.

Other example experiment fields 410 include sample treatment, a time point, a species, a host species, a number of samples, an experiment year, methods, keywords, rights, rights holder, created at, contributor, and/or a submitter.

Each experiment field 410 can be associated with a various data type 420. For example, some experiment fields 410 can be free text (e.g., biological study identification, an experiment tag, an experiment description, and/or a method) or a list of free text items (e.g., related studies, sample treatment, time point, an experiment year, a keyword, rights, rights holder, created at, contributor, contact, and/or submitter). Some experiment fields 410 can be a listing of CV terms (e.g., measurement type, technology type, platform, anatomical entity, cell type, cell line, sample acquisition method, disease, sample disease activity, species, and/or host species). Other experiment fields 410 can be an integer (e.g., number of samples).

Some experiment fields 410 can be mandatory such that a user must enter data associated with these fields when uploading digital data. Other experiment fields 410 can be optional fields of data that may be entered at the discretion of the user uploading the therapeutic and/or biological digital data. Example experiment fields 410 that are mandatory can include a study identification, an experiment tag, a measurement type, a technology type, a platform, and/or a submitter. Example optional fields can include related studies, experiment description, anatomical entity, cell type, cell line, sample acquisition method, disease, sample disease activity, sample treatment, time point, species, host species, number of samples, experiment year, methods, rights, rights holder, created at, contributor, and/or contact. Although, the anatomical entity, cell type, and cell line experiment fields 410 are optional, there are some exceptions. For example, when experiment used both whole blood and isolated T cells, the anatomical entity should contain whole blood and cell type should contain T cell. However, if T cells were isolated from whole blood and only the isolated T cells were used in the experiment, then anatomical entity is empty and the corresponding cell type is a T cell.

The following fields apply to the samples collected for the experiment: anatomical entity, cell type, cell line, sample acquisition method, disease, sample disease activity, sample treatment, time point, species, and/or host species. These fields should indicate the range of possible values for individual samples, as available through sample information sheets and/or design tables. These fields can provide a summary of the available measurements without curating individual sample information.

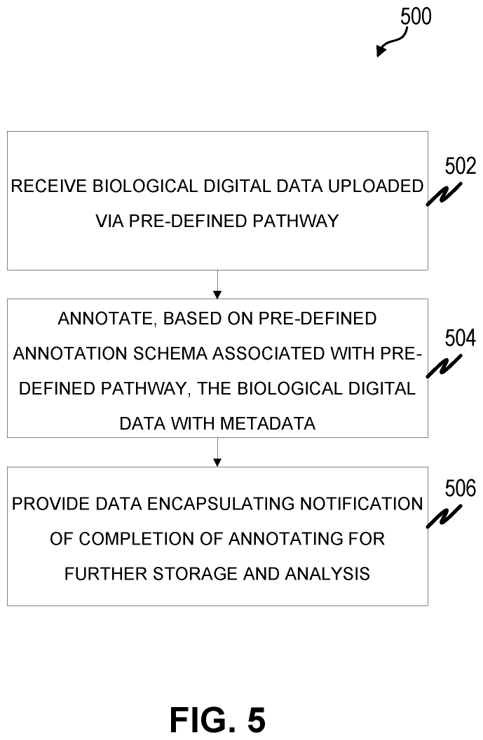

FIG. 5 is a flow chart 500 illustrating a method for managing therapeutic and/or biological digital data. Therapeutic and/or biological digital data uploaded via a pre-defined pathway is received, at 502. Based on a pre-defined annotation schema associated with the pre-defined pathway, the therapeutic and/or biological digital data is annotated, at 504, with metadata. The metadata facilitates storage and identification of the annotated therapeutic and/or biological digital data in a permanent data repository. Data encapsulating a notification is provided, at 506, which indicates completion of the annotating for further storage and analysis.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “computer-readable medium” refers to any computer program product, apparatus and/or device, such as for example magnetic discs, optical disks, memory, and Programmable Logic Devices (PLDs), used to provide machine instructions and/or data to a programmable processor, including a computer-readable medium that receives machine instructions as a computer-readable signal. The term “computer-readable signal” refers to any signal used to provide machine instructions and/or data to a programmable processor. The computer-readable medium can store such machine instructions non-transitorily, such as for example as would a non-transient solid-state memory or a magnetic hard drive or any equivalent storage medium. The computer-readable medium can alternatively or additionally store such machine instructions in a transient manner, for example as would a processor cache or other random access memory associated with one or more physical processor cores.

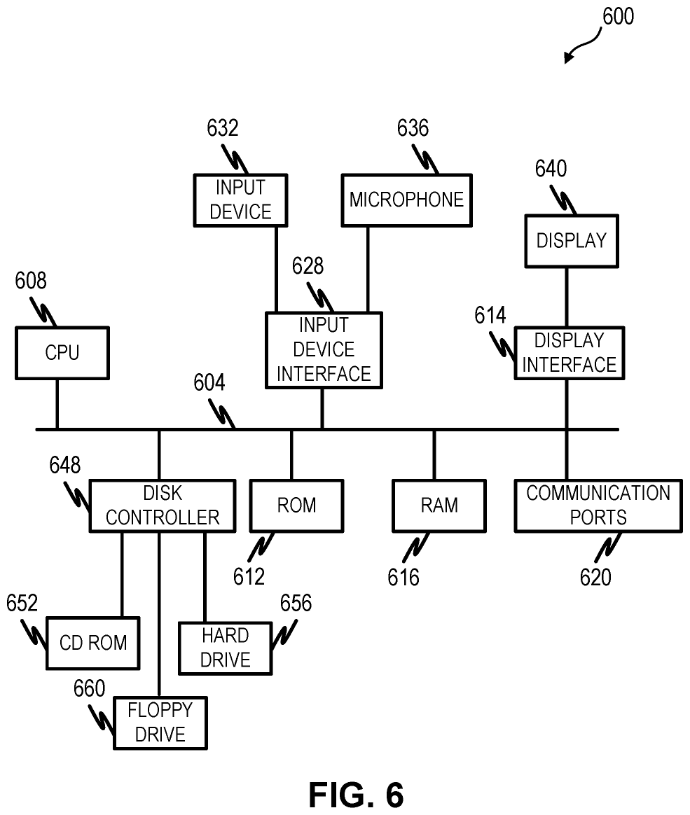

FIG. 6 is a diagram 600 illustrating a sample computing device architecture for implementing various aspects described herein. A bus 604 can serve as the information highway interconnecting the other illustrated components of the hardware. A processing system 608 labeled CPU (central processing unit) (e.g., one or more computer processors/data processors at a given computer or at multiple computers), can perform calculations and logic operations required to execute a program. A non-transitory processor-readable storage medium, such as read only memory (ROM) 612 and random access memory (RAM) 616, can be in communication with the processing system 608 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 648 can interface one or more optional disk drives to the system bus 604. These disk drives can be external or internal floppy disk drives such as 660, external or internal CD-ROM, CD-R, CD-RW or DVD, or solid state drives such as 652, or external or internal hard drives 656. As indicated previously, these various disk drives 652, 656, 660 and disk controllers are optional devices. The system bus 604 can also include at least one communication port 620 to allow for communication with external devices either physically connected to the computing system or available externally through a wired or wireless network. In some cases, the communication port 620 includes or otherwise comprises a network interface.

To provide for interaction with a user, the subject matter described herein can be implemented on a computing device having a display device 640 (e.g., a CRT (cathode ray tube) or LCD (liquid crystal display) monitor) for displaying information obtained from the bus 604 to the user and an input device 632 such as keyboard and/or a pointing device (e.g., a mouse or a trackball) and/or a touchscreen by which the user can provide input to the computer. Other kinds of input devices 632 can be used to provide for interaction with a user as well; for example, feedback provided to the user can be any form of sensory feedback (e.g., visual feedback, auditory feedback by way of a microphone 636, or tactile feedback); and input from the user can be received in any form, including acoustic, speech, or tactile input. In the input device 632 and the microphone 636 can be coupled to and convey information via the bus 604 by way of an input device interface 628. Other computing devices, such as dedicated servers, can omit one or more of the display 640 and display interface 614, the input device 632, the microphone 636, and input device interface 628.

To provide for interaction with a user, the subject matter described herein can be implemented on a computer having a display device (e.g., a CRT (cathode ray tube) or LCD (liquid crystal display) monitor) for displaying information to the user and a keyboard and a pointing device (e.g., a mouse or a trackball) and/or a touchscreen by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an un-recited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

1. A method for managing digital data comprising biological digital data or therapeutic digital data, the method being implemented by one or more data processors forming one or more computing devices and comprising:
receiving digital data comprising biological digital data or therapeutic digital data via a pre-defined pathway;
annotating, based on a pre-defined annotation schema associated with the pre-defined pathway, the digital data with metadata, wherein the metadata facilitates storage and identification of the annotated digital data in a permanent data repository; and
providing data encapsulating a notification of completion of the annotating for further storage and analysis.

2. The method of claim 1, wherein the providing comprises at least one of: causing the notification to be displayed in a graphical user interface on an electronic visual device, loading the data encapsulating the notification into memory, storing the data encapsulating the notification into physical data storage, or transmitting the data encapsulating the notification to a remote computing system.

3. The method of claim 1, wherein the metadata comprises at least one mandatory study field that describes at least one of (i) a biological study identification (ii) a biological study type defining a type of study in drug development, preclinical research, or a clinical trial, (ii) a biological study name, (iii) a biological study description defining the study objectives, protocol, or design, (iv) an organism under study, or (v) a submitter.

4. The method of claim 1, wherein the metadata comprises at least one mandatory experiment field that describes at least one of (i) a biological study identification, (ii) an experiment tag, (iii) an experiment description, (iv) a measurement type, (v) a technology type defining a detection method or technology used to conduct an experiment, (vi) a platform defining a version of the technology type used to conduct the experiment, (vii) a contributor, (viii) a contact defining a primary point of contact for the digital data, or (ix) a submitter of the digital data.

5. The method of claim 1, wherein the metadata comprises at least one optional study field that describes at least one of (i) a study intervention defining a compound or a molecule under study, (ii) a disease under study, (iii) a therapeutic area, (iv) a functional area, (v) a disease area stronghold, (vi) a pathway area stronghold, (vii) a keyword, or (viii) an electronic lab notebook number.

6. The method of claim 1, wherein the metadata comprises at least one optional experiment field that describes at least one of (i) a related study identifier, (ii) an atomical entity defining where samples for an experiment originated, (iii) a cell type classification, (iv) cell line information, (v) a sample acquisition method defining a method or a procedure used to acquire a sample, (vi) a disease under study, (vii) sample disease activity defining status of a disease of the sample, (viii) sample treatment defining an agent used to treat the sample, (ix) a time point defining a sample collection time point, (x) a species under study, (xi) a host species defining a host organism for the study, (xii) a number of sample taken for the experiment, (xiii) a method used to generate the digital data, (xiv) a keyword associated with the digital data, (xv) a rights statement, (xvi) a rights holder, (xvii) a creation location defining a location where the digital data was generated, or (xviii) a contributor to the digital data.

7. The method of claim 1, wherein the annotating comprises:
determining a data format of the digital data
consolidating and converting, based on the data format, the digital data to a parsable, human readable text file format; and
assigning the metadata to the parsable, human readable text file format.

8. The method of claim 1, further comprising transferring and storing the digital data in a read-only format to the permanent data repository.

9. The method of claim 1, wherein the pre-defined pathway points to a hierarchical data folder in an intermediary data repository and wherein the metadata is associated with the hierarchical folder.

10. The method of claim 1, wherein the notification informs an administrator to transfer the digital data to the permanent data repository.

11. The method of claim 1, wherein data stored in the permanent data repository cannot be modified, deleted, or overwritten.

12. The method of claim 1, further comprising providing the digital data in a read-only format to a graphical user interface for inspection.

13. The method of claim 1, wherein the metadata is defined by a user when uploading the digital data using the pre-defined pathway.

14. The method of claim 1, wherein the digital data comprises biological digital data including at least one of bio therapeutic data, biotechnological data, molecular data, biomarker data, transcriptional data, phenome data, or image data.

15. A method for managing digital data, the method comprising:
a step for receiving digital data comprising therapeutic digital data or biological digital data uploaded via a pre-defined pathway;
a step for annotating, based on a pre-defined annotation schema associated with the pre-defined pathway, the digital data with metadata, wherein the metadata facilitates storage and identification of the annotated digital data in a permanent data repository; and
a step for providing data encapsulating a notification of completion of the annotating for further storage and analysis.

16. A system for managing biological digital data comprising:
at least one data processor; and
memory storing instructions, which when executed by at least one computing device, result in operations for implementing a method as in claim 1.

17. A non-transitory computer program product storing instructions which, when executed by at least one data processor forming part of at least one computing device, implement a method as in claim 1.
